(19) United States
(12) Patent Application Publication
(10) Pub. No.: US 2006/0193874 A1
(43) Pub. Date: Aug. 31, 2006

(54) Vaccines for proliferative ileitis and methods of making and using the same

(76) Inventor: Lynn A. Jones, Tucson, AZ (US)

Correspondence Address: INTERVET INC.;PATENT DEPARTMENT, PO BOX 318, MILLSBORO, DE, 19966-0318, US

(21) Appl. No.: 11/181,484

(22) Filed: Jul. 14, 2005

Related U.S. Application Data
(62) Division of application No. 09/677,108, filed on Sep. 29, 2000, now abandoned.

(51) Int. Cl. A61K 39/02

(52) U.S. Cl. 424/234.1

(57) ABSTRACT

A proliferative ileitis vaccine comprising tissue culture grown Lawsonia intracellularis and methods of making said vaccines. Proliferative ileitis vaccines described include those containing whole L. intracellularis, extracts of L. intracellularis, protective immunogenic subunits of L. intracellularis, recombinant immunogens of L. intracellularis and naked DNA of L. intracellularis. The vaccines of this invention may be inactivated or modified live and contain adjuvants and/or stabilizers. The vaccines of this invention may be in a liquid or lyophilized form. Also disclosed are monoclonal antibodies which neutralize the growth of L. intracellularis and which may be used for diagnosing proliferative ileitis as well as for quantitating antigen during vaccine production.

### BACKGROUND OF INVENTION

1. Field of the Invention

The present invention relates generally to the preparation of vaccines and diagnostics for protecting pigs from proliferative ileitis caused by Lawsonia intracellularis.

2. Brief Description of the Prior Art

Porcine proliferative ileitis, sometimes referred to as porcine proliferative enteritis, is a major problem for the U.S. swine industry. Proliferative ileitis is an intestinal disease complex of pigs characterized by crypt hyperplasia and by the presence of intracellular campylobacter-like organisms. Recognition of the disease has increased dramatically in the past ten years, with the incidence ranging as high as 20% and losses estimated at $50 million annually in the U.S. alone. Especially alarming is the apparent increase in incidence among the seed stock industry. The disease has been found worldwide and usually affects post-weaning pigs between six and twenty weeks of age. The clinical signs of pigs affected with proliferative ileitis include intermittent diarrhea, anorexia, marked dullness and apathy, and a wasting syndrome. Death is not uncommon and is frequently associated with hemorrhage effects on intestines. Four different forms of the disease have been described, but the majority of the literature groups the lesions into two forms, acute and chronic, sometimes referred to as necrotic.

In the acute phase of the disease, macroscopic lesions are characterized by the presence of mesenteric and subserosal edema and pronounced reticulation of the serosa. The mucosa and muscle layers are thickened, with the mucosa forming deep longitudinal or transverse folds. The ridges of the folds are often hyperemic with some hemorrhage. Ulceration of the mucosa is occasionally evident with areas of viable mucosa adjacent to the lesions. At a microscopic level, acute lesions are characterized by an acute non-specific inflammation accompanied by hyperemia and edema in the lamina propria, by proliferation of epithelial cells, and by hypertrophy of the ileal musculature. The villi and intestinal crypt structure become irregular, with epithelial cell dysplasia. Crypt abscesses form as crypts become branched and filled with inflammatory cells. The villi and lamina propria become swollen due to the presence of edema and inflammatory cells. The submucosa usually contains lymphatic tissue nodules. The muscle of the ileum, especially the circular layers, may become hypertrophied.

In the chronic phase of the disease, macroscopic lesions are characterized by thickening of the lower small intestine. In some cases, inflammation and epithelial cell necrosis result in the formation of a fibrinous exudate. The mucosa frequently shows hyperemia and hemorrhage with occasional ulceration. The intestine is usually thickened throughout the lower small intestine, but mucosal thickening is discontinuous, so that the mucosa is always thinner along the mesenteric attachment. Sometimes, the lumen contains a formed blood clot and the colon may contain tarry feces of mixed blood and digesta. The mucosal surface of the affected small intestine may show little gross damage except for the thickening. Erosions, bleeding points, and ulcers are usually not observed. In addition, the regional lymph nodes are markedly swollen. Microscopic lesions in the chronic stage are characterized by a transmural inflammatory reaction. The mucosa is more distorted with long irregular epitheloid projections. Crypts branch, and produce fissures in the mucosa at the borders of the hyperplastic areas. Ulcers may be seen. Crypt abcesses are also prominent in these areas. Intracellular bacteria, shaped as curved rods, are usually present in the crypt epithelium. The hyperplastic epithelial cells are columnar. Regenerative tissue contains irregularly formed epithelial cells with a strongly basophilic cytoplasm. Goblet cells are usually infrequent in regeneration areas, but numerous in hyperplastic areas. The lamina propria typically exhibits inflammatory cell infiltration. Granulomas are occasionally present in ulcerated areas. The submucosa contains dilated lymphatic vessels and fluid-filled tissue spaces. The muscle layers are hypertrophied, with alterations due to the presence of inflammatory cells and fibrin.

The presence of intracellular bacteria in the crypt epithelium of afflicted animals confirms a bacterial etiology for the disease. Although bacteria isolated from such animals are morphologically similar to Campylobacter spp, hybridization studies and reproduction experiments using various Campylobacter strains have demonstrated that this organism is not the etiological agent. Joens and Glock (U.S. Pat. No. 5,610,059) describe and claim the isolation and characterization of porcine proliferative enteritis organism and reproduction of the disease using the organism, now known as Lawsonia intracellularis.

Effective proliferative ileitis control measures have been limited. A basic trial-and-error therapeutic regimen, which includes the use of oral and parenteral broad-spectrum antibiotics, antihistamines, corticosteroids, nitroimidazole, and B vitamins, usually becomes quite costly and typically proves ineffective. There is no report to date of any attempts to prepare a vaccine to control this disease problem.

### SUMMARY OF THE DRAWINGS

FIG. 1 compares the antibody response produced by vaccination of pigs with a vaccine comprising L. intracellularis with non-vaccinated control pigs.

FIG. 2 is a Western blot of sera from convalescent pigs reacted to an extract of L. intracellularis.

FIG. 3 is a Western blot of sera from L. intracellularis vaccinated pigs reacted to an extract of L. intracellularis.

FIG. 4 is a Western blot of monoclonal antibodies reacted to an extract of L. intracellularis.

FIG. 5 is a Western blot of monoclonal antibodies reacted to an extract of L. intracellularis.

FIG. 6 is a dose-dependent L. intracellularis neutralization curve comparing monoclonal antibody 5A2 and V or 4 with a non-neutralizing monoclonal antibody.

### SUMMARY OF THE INVENTION

An intracellular organism isolated from pigs showing clinical proliferative ileitis, which was the subject of U.S. Pat. No. 5,610,059 which was referred to previously as PPE-causing agent, ileitis agent, IL-A, (ATCC No. 55370) has now been named Lawsonia intracellularis. The initial isolate was shown to reproduce the disease of proliferative ileitis. Since this initial report, at least four additional isolates have been obtained and shown to demonstrate the same growth characteristics as ATCC 55370 confirming that ATCC No. 55370 is the prototype organism for a new genus and species of bacteria.

It is the object of the present invention to provide improved vaccines that protect against the disease complex collectively called proliferative ileitis caused by L. intracellularis. Such vaccines can be of any type, including but not limited to inactivated, live, modified live, subunit, recombinant, vector-delivered recombinant or naked DNA.

It is another object of this invention to identify antigens leading to the identification of immunogens of L. intracellularis, which can be useful in preparation of subunit or recombinant vaccines. By the term “immunogen” is meant the antigen(s), or portions thereof, which produce a protective response in the host animal when injected into said host animal or which produces an antibody which inhibits infection by L. intracellularis in vivo or in vitro. By the term “identify” is meant searching for and finding the specific antigen(s), characterizing their molecular weight, and demonstrating their ability to function as an immunogen. Some of the immunogens described herein produce monoclonal antibodies (MOAB) that have demonstrated an ability to neutralize the growth of L. intracellularis. Monoclonal antibodies of this invention react with antigens having molecular weights ranging from 15 kilodaltons (kDa) to 250 kDa, more specifically, having molecular weights of 21, 31, 41, 43-44, 60, 71, and ≧115 (115 to 250) kDa. As would be realized, sequencing of these antigens will provide information necessary to develop PCR probes for detecting L. intracellularis in tissue samples as well as monitoring growth in tissue.

It is a final object of this invention to provide monoclonal antibodies (MoAbs) which can be used for diagnosis of proliferative ileitis or for development of antigen quantitation tests. Some of these MoAbs have been demonstrated to be neutralizing in that they inhibit the development of the cytopathic effect (CPE) when L. intracellularis is grown in Henle cells. By CPE is meant that the tissue culture cell monolayers are so adversely affected by the L. intracellularis that the cells die and slough off of the surface of the vessel onto which they are attached. Useful MoAbs include but are not limited to clones 2A2, 2B6, 5A2, 3A1, 2C1, 3D4, and 1C2, which correlate to antigens having molecular weights of 43-44, 60 kDa, 41 and 43-44 kDa, 41 kDa, 60 kDa, 71 kDa, and ≧115 kDa, respectively. Of these, MoAbs 2A2(detecting an antigen with a molecular weight between 43 and 44 kDa), 5A2 (detecting an antigen with molecular weights of 41, 43-44 kDa), 3A1 (detecting an antigen with a molecular weight of 41 kDa), 3D4 (detecting an antigen with a molecular weight of 71 kDa) and 1C2 (detecting an antigen with a molecular weight equal to or greater than 115 kDa) are neutralizing and have the most potential for use in diagnostic and antigen quantitation assays as well as for use in identifying target vaccine antigens including recombinant antigens. These studies indicate that an effective L. intracellularis vaccine must produce an immune response, possibly antibodies, to one or more of the antigens with molecular weights of 41 kDa, 43-44 kDa, 71 kDa and 115 kDa or greater.

### DETAILED DESCRIPTION OF THE INVENTION

As set forth above, the present invention is directed to improved vaccines which protect against the disease complex collectively called proliferative ileitis which is caused by the prototype organism, L. intracellularis ATCC No. 55370 and all strains and mutants thereof which have similar immunogenic characteristics. Similar immunogenic characteristics are defined as the capability to cross-react immunologically with ATCC 55370 and/or the ability to protect pigs from proliferative ileitis.

The improved vaccines include but are not limited to conventional types of vaccines such as modified live vaccines, attenuated vaccines, inactivated vaccines, subunit vaccines, and recombinant type vaccines such as vaccines in which one or more protective antigens from L. intracellularis are produced by an organism other than L. intracellularis and are used in crude or purified form from this organism or recombinant vaccines in which one or more protective antigens are carried by a live vector such as another live or modified live bacteria, a live or modified live virus, a live prokaryotic cell or some other type of live organism. Also, covered by this invention are vaccines containing naked DNA encoding for protective immunogens of L. intracellularis, said protective immunogens being described herein.

It is understood by those skilled in the art that neither the growth of an organism nor the identification of antigen(s) from that organism can assure that a vaccine can be produced. All organisms, especially bacteria, contain thousands of antigens, some of which produce detrimental responses in the host when used in vaccines, some of which produce no response of significance and a few (generally less than 5) produce an immune response which is truly involved in protection of animals. These immunogens are protective if they are prepared in an unaltered form (structure, shape and sequence are intact and they are presented to the animal by the animal's immune system and if the animal's immune system can develop a high enough immune response to produce protection (immunogenically effective amount). In the preparation of vaccines for protection animals, it is critical to maintain the structure and configuration of the immunogens, especially in fragile bacterial such as L. intracellularis, to produce a high enough level of immunogen(s) (antigenic mass), and to include adjuvants or immunomodulators, especially in inactivated, recombinant or subunit vaccines in order to be successful. In the preparation of modified live, attenuated or live vaccines, it is critical to maintain the immunogens of the fragile organisms as well. Most of the time, this is accomplished by addition of stabilizers, lyophilization, freeze drying or vitrification. However, even in this instance, using specific methods of growth which conserve the structure of the immunogen and adding the correct stabilizers are critical.

U.S. Pat. No. 5,610,059 (Joens et al) describes general methods for preparing inactivated and modified or attenuated vaccines. The present invention describes improved methods for preparing inactivated vaccines for L. intracellularis as described herein and comprise the steps of: 1) growing the L. intracellularis organism in a susceptible tissue culture utilizing a vessel including but not limited to flasks, roller bottles, and bioreactors containing media, and using the vessel surface, beads or other substrates for tissue culture cell attachment or growing the L. intracellularis in suspensions of tissue culture cells in bioreactors; 2) harvesting the L. intracellularis by removing the grown L. intracellularis organisms from the tissue culture vessel; 3) inactivating the L. intracellularis organisms without damaging the immunogens; and 4) adjuvanting the inactivated L. intracellularis organisms. The term “susceptible” means that the tissue culture has been specifically selected or cloned to grow the L. intracellularis organism and express its immunogens such that the immunogens are not modified or altered and a high antigenic mass of organisms is produced.

The susceptible tissue culture useful for growing L. intracellularis may be selected from the group consisting of simian cells, murine cells, rat cells, canine cells, feline cells, hamster cells, human cells, equine cells, fish cells, hamster cells, bovine cells and swine cells. More specifically, the tissue cultures may be selected from the group consisting of Vero cells, MA-104 cells, 9009B cells (CRL 11302), Buffalo Green Monkey Kidney cells (BGM), McCoy cells, L-929 cells, Madin Darby Canine Kidney cells (MDCK), Cutter Laboratories Dog Kidney cells (CLDK), Crandell cells (CRFK), Embryonic Bovine Turbinate cells (EBT), EHTh cells, BFSp cells, Swine Testicle cells (ST), Swine Kidney cells (SK), PK-15 cells, Baby Hamster Kidney cells (BHK-21), Henle 407 cells and rat intestinal epithelial cells (IEC). Preferred cells include but are not limited to Vero cells, MDCK cells, Henle 407 cells and IEC cells, said cells having been shown to produce a significant amount of cytopathic effect (CPE) without modification of the organism or the cell. Growth in other cells has been demonstrated by a polymerase chain reaction (PCR) technique. It is expected that any cell, which grows L. intracellularis, with or without CPE, is a susceptible tissue culture cell, which is acceptable for vaccine production. The most preferred cells for growth of L. intracellularis without modification of the immunogens would be clones of the above tissue culture cells which are susceptible and specifically grow the L. intracellularis to a high antigenic mass in a short period of time (within 10 days).

The improved process of growing L. intracellularis starts with inoculating a susceptible tissue culture, in suspension, as a cell sheet attached to a vessel wall or bead substrate or as a confluent monolayer attached to a vessel or bead substrate in the presence of a media which supports the growth of the organism. The media for growth can be any of a typical tissue culture media such as, but not limited to Minimal Essential Medium (MEM). The infected tissue culture is then incubated at a temperature which is able to support the growth of L. intracellularis, generally between 30° C. and 40° C., preferably between 34° C. and 38° C., more preferably between 35° C. and 37° C., for a period of time to enable the organism to produce the required antigenic mass. The period of time can vary between 1 and 30 days with the required antigenic mass being produced preferably between 5 and 10 days of incubation. Growth can be measured by numerous means including fluorescent antibody staining (FA), indirect fluorescent antibody staining (IFA), PCR, and enzyme linked immunosorbant assay (ELISA). The latter techniques can be used to quantitate the specific antigenic mass to have assurance that the vaccine contains adequate immunogen to produce protection. Growth is complete when the maximum antigenic mass has been produced. After growth is complete, the culture is harvested.

The harvesting process requires removal of the fluids from the vessel. Said fluids may contain cell debris or whole tissue culture cells in addition to the L. intracellularis. The harvesting step may include a freeze-thaw step, treatment with enzymes or detergents, or treatment with high pressures in order to break open the tissue culture cells to release the L. intracellularis organisms and/or antigenic mass. Additionally, the harvesting step may include concentration using art-known techniques such as centrifugation, column chromatography or ultrafiltration.

Inactivation of L. intracellularis can be accomplished by treating the organism with binary ethyleneimine, beta-propiolactone, formaldehyde, heat, detergents or any other art-known agent which can inactivate organisms without damaging the immunogenicity. In this respect, immunogenicity is defined as the ability of a vaccine to protect pigs against clinical or subclinical signs of proliferative ileitis caused by L. intracellularis or the ability of antibodies to the immunogens to inhibit growth of the L. intracellularis. Preferred antibodies raised by the vaccines of the present invention include but are not limited to those reacting with antigens with molecular weights of 41, 43-44, 60, 71 and greater than or equal to 115 kDa.

Adjuvants and/or immunomodulators are typically added to inactivated vaccine antigens to stimulate a higher immune response and provide for a stronger protection. Such adjuvants which can be used with L. intracellularis may be selected from the group consisting of bacterial-based adjuvants including Freunds Complete adjuvant, Freunds Incomplete Adjuvant, Corynebacterium, Propionibacterium and Mycobacterium; polymer based adjuvants such as POLYGEN®, Carbopol®, Havlogen®, CARBIGEN®, methacrylates, DEAE Dextran and Dextran sulfate; oil adjuvants such as EMULSIGEN®, EMULSIGEN PLUS® and EMUGEN®; animal oils such as squalane or squalene; mineral oils such as Drakeol and Montanides; vegetable oil such as peanut oil; block co-polymers; aluminum salts such as aluminum hydroxide and aluminum phosphate; poxvirus proteins such as Baypamun; Avridine; Lipid A; triterpenoid glycosides such as saponin, Quil A, and QS21; detergents such as Tween-80, Span 20 and Pluronics; interleukins such as monokines and interferons; liposomes ISCOMS; synthetic glycopeptides such as muramyl dipeptides and derivatives of cholera toxin, or combinations of the above. Preferred adjuvants for use with fragile organisms such as L. intracellularis include but are not limited to POLYGEN™, ethylene maleic anhydride type adjuvants, ethylene maleic acid polymers, CARBIGEN®, HAVLOGEN®, EMULSIGEN®, POLYGEN™, synthetic glycopeptides such as BAY R1005, certain aluminum salts, immunomodulators, toxins and combinations thereof. Such preferred adjuvants are selected on the basis of their lack of toxicity and lack of presence of detergents, which tend to disrupt fragile organisms and immunogens.

Alternatively, this invention describes a vaccine including modified live or attenuated L. intracellularis organisms. In order to produce this type of vaccine, the L. intracellularis is attenuated by multiple passage in tissue culture or treatment with mutagens to produce stable mutations that produce an L. intracellularis organism which is live, which is not pathogenic for swine and which has maintained the immunogens intact. Multiple passaging in tissue culture, preferably in non-swine tissue culture, would be expected to produce an attenuated L. intracellularis. Another procedure, which produces stable mutants is to treat the L. intracellularis with a mutagen such as N-methyl nitrosoguanidine or acridine orange and selecting the L. intracellularis organisms which are non-pathogenic for swine. In this context, the term “non-pathogenic” means that the L. intracellularis replicates in swine but will not reproduce clinical or subclinical signs of disease in swine. Alternatively, modified live or attenuated L. intracellularis organisms can be produced by using the art-known techniques of transposon-induced mutagenisis. Methods for producing modified live or attenuated L. intracellularis vaccines include the steps of: 1) growing the modified live or attenuated L. intracellularis in a susceptible tissue culture in some type of vessel including but not limited to flasks, roller bottles and bioreactors, containing media, and using the vessel surface, beads or other substrates for tissue culture cell attachment or growing the modified live attenuated L. intracellularis in suspensions of tissue culture cells in bioreactors; 2) harvesting the L. intracellularis by removing the grown L. intracellularis organisms from the tissue culture vessel; 3) stabilizing the live L. intracellularis, if necessary; and 4) alternatively, adjuvanting the live L. intracellularis.

The steps of growing and harvesting are similar to those described previously. The step of stabilizing the L. intracellularis can include drying such as freeze-thaw, lyophilization, vitrification or stabilization in a liquid form wherein polymer or sugar stabilizers or other types of liquid stabilizers may be added.

The step of adjuvanting is similar to that described previously and may include any of the above-mentioned adjuvants.

Subunit vaccines are vaccines which contain a portion of the organism but less than the whole organism. Subunit vaccines may contain as few as 1 immunogen. These types of vaccines can be prepared by any of the following means. They can be prepared whenever a vaccine prepared from the whole organism contains detrimental antigens. They can be prepared from the whole organism, which is ineffective because the immunogen is hidden or masked from the animal's immune system by dominant interfering antigens. They can be prepared whenever preparation of a subunit is more commercially feasible or whenever a purer form of vaccine is desired. Subunit vaccines can be made by simply extraction of the L. intracellularis organism and saving the portion that contains the immunogen(s). Subunit vaccines are prepared using the steps of: 1) growing the L. intracellularis in a susceptible tissue culture in some type of vessel including but not limited to flasks, roller bottles and bioreactors, containing media, and using the vessel surface, beads or other substrates for tissue culture cell attachment or growing the L. intracellularis in suspensions of tissue culture cells in bioreactors; 2) harvesting the L. intracellularis by removing the grown L. intracellularis organisms from the tissue culture vessel; 3) extracting the L. intracellularis by treating with detergents, enzymes or freeze-thaw; high pressure such as with a French Press, or any other method which will break open the tissue culture cells as well as the L. intracellularis; 4) purifying the protective antigens (immunogens) in order to remove components which interfere with immunogenicity to produce a subunit; and 5) adjuvanting the subunit. The steps of growing, harvesting and adjuvanting are similar to those described above. The step of purifying the immunogen(s) includes but is not limited to: 1) identifying the immunogens of L. intracellularis; and 2) removing the immunogen(s) by art-known techniques including but not limited to column chromatography, ultrafiltration, centrifugation, electrophoresis and differential centrifugation. Target antigens for subunit vaccines are those L intracellularis proteins that have molecular weights of 41, 43-44, 60 or 71 and are equal to or greater than 115 kDa.

The improved conventional vaccines described herein can be administered intramuscularly, subcutaneously, intranasally, intradermally, orally, intravenously or topically using a carrier.

This invention also describes the preparation of a recombinant vaccine. The most critical step of preparing a recombinant vaccine comprises the identification of one or more target immunogens which are then sequenced and expressed, said sequences being inserted into production vectors or vaccine vectors. The term “identification” as used in this context means the demonstration of the presence of a specific immunogen with a known molecular weight. The term “production vector” is used herein to describe organisms such as bacteria, viruses or eukaryotic cells which have the sequence(s) encoding for the target immunogen(s) inserted into them such that large amounts of the target immunogen(s) are expressed by the vectors. The target immunogen(s) can either be purified away from the production vector or used as part of the production vector. Examples of such production vectors are: E. coli, Salmonella, baculovirus, transfected tissue culture cells, adenoviruses, retroviruses and yeast. The term “vaccine vector” is used herein to mean a live or inactivated production vector which is used to deliver the target immunogen(s) to the animal being vaccinated. Examples of vaccine vectors are Salmonella, E. coli, adenovirus, or any other live organisms which can receive foreign DNA, express it without changing the immunogenicity of the immunogen and replicate within the animal receiving the vaccine.

One method of preparing a recombinant vaccine comprises the steps of: 1) identifying target immunogens; 2) constructing and screening a L. intracellularis genomic library; 3) identifying the recombinant clones producing proteins of L. intracellularis; 4) identifying the genes encoding immunoreactive epitopes; 5) expressing the immunoreactive epitopes via an expression vector or a production vector; and 6) formulating the recombinant target immunogen(s) or the live production vectors into a vaccine.

A second method of preparing a recombinant vaccine comprises the steps of:

1) preparing MoAbs to L. intracellularis;

2) identifying MoAbs which neutralize the growth of L. intracellularis;

3) identifying the antigens which are detected by the MoAbs as target immunogens;

4) sequencing the target immunogens;

5) expressing the target immunogens in one or more expression or production vectors to produce recombinant immunogens; and

6) formulating the recombinant immunogen(s) or the live production vectors into a vaccine.

The expressed immunogen(s) can be recovered from the expression vector, used in a crude form or, alternatively, purified, and then adjuvanted as described previously. Additionally, the expression vector can be administered as a live vaccine delivering the immunogen(s) to the host and allowing replication to increase the amount of immunogen(s) circulating in the pig. The latter system has an advantage in that the vaccine can be more easily administered orally, either directly or in the drinking water. Additionally, it could be delivered intranasally, intramuscularly, subcutaneously or topically with the use of carriers. Alternatively, the naked DNA sequence of the target immunogen(s) can be used as vaccines.

All of the recombinant or naked DNA vaccines described herein can be administered intramuscularly, subcutanteously, intradermally, intranasally, intraveneously, orally, or topically if formulated using art-known techniques.

As indicated previously, the critical step of producing a subunit, recombinant or naked DNA vaccine for L. intracellularis is identification of the target immunogen(s). This invention describes target immunogens of L. intracellularis which have been identified and would be expected to be useful for subunit, recombinant or naked DNA vaccines. Such target immunogens are also useful in diagnosing proliferative ileitis in diseased swine or quantitating antigens in a vaccine using such techniques as the quantitative PCR described herein or the ELISA technology.

One successful method for identifying the target immunogens of L. intracellularis utilized electrophoresis and the method of Western blotting of L. intracellularis antigen extracts with serum from convalescent pigs (pigs which have recovered from Proliferative ileitis) and looking for antigens consistently recognized by these sera. A second successful method for identifying the immunogen(s) of L. intracellularis was to observe key antibodies which were developed by pigs vaccinated with a vaccine prepared as described above and which showed protection against a challenge with a heterologous strain of L. intracellularis. Using both of these methods, antigens having molecular weights of 21 kDa, 31 kDa, 41 kDa, 43-44 kDa, 60 kDa, 71 kDa and ≧115 kDa have been identified as target immunogens. These antigens can be useful in preparation of subunit, recombinant or naked DNA vaccines or used in developing diagnostics for proliferative ileitis or antigen quantitation methods.

Proof of the functionality of the above-listed antigens, and their identification as target immunogens for subunit, recombinant and naked DNA vaccine production was conducted as follows. First, there is provided herein the preparation of monoclonal antibodies (MoAbs) recognizing the 31 kDa, 41 kDa, 43-44 kDa and 60 kDa, 4344 kDa, 60 kDa, 71 kDa and 115 kDa proteins using art-known techniques. The hybridomas producing these MoAbs were identified as 6B1, 3A1, 2A2, 2B6, 5A2, 2C1, 3D4 and 1C2 respectively. A confirmation that these antigens were indeed immunogens was demonstrated by proving their functionality (i.e., the ability of the MoAbs produced thereto to neutralize the development of L. intracellularis cytopathic effect in tissue culture). Indeed, the 41, 43-44, 71 and 115 or greater kDa antigens listed produced neutralizing MoAbs. Therefore, any or all of these immunogens are candidates for subunit, recombinant or naked DNA vaccines. The monoclonal antibodies described herein would be expected to be useful for diagnosing proliferative ileitis or for development quantitative PCR or ELISA assays for detection of antigen in vaccines as well as the detection of L. intracellularis in tissue samples, fecal samples, or circulating in the blood of animals.

Further, for the development of subunit, recombinant or naked DNA vaccines and after identifying the target immunogens, a genomic library was produced by extraction of L. intracellularis chromosomal DNA using conventional SDS/proteinase K lysis of the bacterial cells, followed by chloroform isoamyl alcohol extractions and ethanol precipitation. Purified DNA was partially digested with restriction endonuclease Sau 3A1 and the fragments were electrophoretically separated through a low melt agarose gel containing ethidium bromide. Fragments three to six kb in length were excised from the gel, purified and ligated in the BamH I site of vector μ-Zap. Ligation products were in vitro packaged using Gigapack II Gold λ phage extracts and recombinant phages transduced into E. coli XL-1 Blue MRF′ as directed by the manufacturer. The transfected bacteria were serially diluted in melted top agar containing X-gal and IPTG and spread on pre-warmed solid agar. Plates were incubated 4 to 8 hours to allow plaque development. Plaques were examined for optimal density and β-galactosidase activity (indicative of non-recombinant phage and the background frequency noted). The lawns and plaques were overlaid with IPTG soaked Immobilon and incubated an additional 5 hours. The membranes were removed and preincubated in blocking solution prior to screening with mono or polyclonal antibodies. Duplicate colony lifts were prepared to allow screening with all antibody stocks. Immunoreactive plaques were lifted and mixed with fresh E. coli XL-1 Blue MRF′ and plaques generated as before. Colony blots and screening with the appropriate antibodies were repeated to ensure clonality and stability of reactive plaques. Stable clonal immunoreactive plaques and fresh bacteria were mixed to generate lysate for western analysis to determine the molecular weight of the antigen harboring the reactive epitope. Immuno-reactive plaques were expanded and converted to single-stranded phagemid DNA (pBK-CMV) by infecting E. coli XL-1 Blue MRF′ with M13 derived helper phage RE704. Recombinant helper phages were transduced into the non-permissive E. coli host XLOLR and plated on selective media to recover double stranded plasmid DNA. Plasmid DNA was extracted via alkaline lysis and digested with restriction endonucleases Pst I and IcoR I to determine the size of insert of DNA. The Pst I/EcoR I fragment was gel purified, digoxygenin (dig) labeled and employed in Southern analysis to determine the source of the DNA (IEC or L. intracellularis). Probes which are bound to IEC infected DNA alone were further characterized by sequencing insert DNA using vector borne M13 forward and reverse primers. Subsequent sequencing reactions were catalyzed by primer walking. If the insert DNA was large (>3 kb) subclonal analysis was employed to focus sequencing efforts. Sequence data were screened for homology to related proteins using the BLASTp algorithm. Similarly, sequence data were analyzed for presence of open reading frames using DNA Stider and ORFs screened using BLASTp searches. Extraction and purification of uncontaminated L. intracellularis was difficult as host cell DNA was ubiquitously present in all L. intracellularis prepared. Therefore, to facilitate identification of immunoreactive clones, immunoscreening of recombinant clones using both mono and poly-clonal antibodies were performed as clones were generated. Verification of L. intracellularis as the source of DNA encoding the reactive epitope was accomplished via PCR and Southern Blot analysis.

In order to construct and screen the L. intracellularis genomic library, the phage expression vector y-Zap was used. This library allows for the identification of bacterial sequences encoding immuno-reactive epitopes which are later compared to the target immunogens described previously.

Isolation and identification of genes encoding immunoreactive epitopes via library screening can be problematic as the size of the L. intracellularis genome is unknown. More importantly, construction of a L. intracellularis genomic library required extraction of semi-purified L. intracellularis which would likely result in the generation of DNA composite of host cell and bacterial chromosomes. As such, identification and isolation of L. intracellularis genomic sequences encoding immunoreactive epitopes was facilitated using inverse PCR.

The first step toward identifying open reading frames (ORFs) encoding immuno-reactive antigens involved purification of immuno-reactive antigens via preparatory SDS-PAGE. The separated factors were transferred to Immobilon and total proteins visualized by amino black staining. Proteins migrating at the appropriate size were excised and subjected to Edman degredation to obtain N-terminal sequence data. At least thirty amino acids will be sequenced to permit identification of residues encoded by a few codons, thereby allowing synthesis of low degeneracy primers. The degenerate primers were used to amplify product from L. intracellularis genomic DNA and host DNA (control) in a touchdown PCR. Ampification products were analyzed on 2% agarose gels. Products of the expected size were gel purified, cloned into a T-tailed pBluescript vector and sequenced using vector borne M13 forward and reverse primers. Sequence data were analyzed for an ORF and translated using DNA Strider. The translated sequence is compared to the amino terminal sequence to verify the PCR product as the coding origin. The PCR product was then digoxygenin labeled and used to probe the L. intracellularis genomic DNA and host cell DNA (control) cut with a battery of restriction endonucleases. Hybridization profiles were analyzed to identify digestion conditions which generate fragments amplifiable by TaqPlus mediated inverse PCR and complete downstream Orfs (size of desired restriction fragment depends on the size of the immuno-reactive antigen; i.e., 20 kDa antigen encoded by ˜500 bp ORF, 60 kDa antigen encoded by ˜1500 kb ORF). Once the source of the PCR product (L. intracellularis) and optimal digestion patterns were determined, L. intracellularis genomic DNA were digested with the desired restriction endonuclease, the fragments separated through a low melt agarose gel, and fragments of desired size (as determined by above Southern analysis) were excised and purified. The purified fragments were diluted to a concentration, which favors intra-molecular ligation and covalently closed using T4 DNA Ligase. Ligation products were used as template for inverse PCR. The inverse PCR utilized primers derived from initial PCR product sequence data. The primers were fashioned to prime polymerization of DNA upstream of the 5′ end of the N-terminal coding sequence and downstream of the 3′ end of the N-terminal coding sequence. Inverse PCR makes use of the TaqPlus Long PCR System to produce long accurate amplicons. Products of the inverse PCR were separated through agarose gels stained with ethidium bromide and analyzed. Verification of the expected size products as those derived from L. intracellularis sequences upstream and downstream of the N-terminal encoding sequence employ restriction mapping. The PCR product is cloned into a T-tailed pBluescript vector and sequenced using vector borne M13 forward and reverse primers. Subsequent sequencing reactions were catalyzed by primer walking. Immunoreactivity of epitopes encoded by the cloned DNA were assessed using western analysis of lysates derived from IPTG induced recombinant clones. When the insert DNA is large (>3 kb) subclonal analysis is employed to focus sequencing efforts on regions encoding immuno-reactive epitopes. Sequence data were assembled with N-terminal encoding DNA sequence data using Sequencer contig assembly software. Identification of the junction separating un-linked sequences (artificially linked by intra-molecular ligation) was accomplished by identifying the restriction site used for intra-molecular ligation. Once assembled, sequence data were screened for homology to related proteins using the BLASTp algorithm. Similarly, sequence data were analyzed for presence of open reading frames using DNA Strider and ORFs screened using BLASTp searches. Production of purified recombinant protein antigen to be used in preparing vaccines for immunization studies is accomplished by incorporating the ORF encoding the immunoreactive epitope into appropriate His tag or calmodulin tag vectors.

Recombinant vaccine preparation has been described previously. Testing the efficacy of said vaccines is accomplished by methods already used for conventional vaccines which include vaccinating pigs and measuring the antibody response by serum neutralization. Data presented in the examples to follow, indicate that the humoral response is very important in the development of immunity against proliferative ileitis in pigs. The humoral antibody response is measured and then the pigs are challenged with a virulent strain of L. intracellularis. One or more of the target immunogens or epitopes from said target immunogens have produced an antibody response in animals and, therefore, similarly vaccinated pigs will be protected as they are with a conventional vaccine.

As indicated previously, naked DNA vaccines are easily prepared once the DNA sequences encoding for the target immunogen(s) have been identified. Naked DNA vaccines utilize pure DNA which may be administered intramuscularly, subcutaneously, intravenously, intranasally, intradermally, orally and topically using known techniques and carriers.

When preparing vaccines of a conventional or recombinant type, quantitation of the antigenic mass is critical, especially when the immunogen is in an inactivated or subunit form. Antigen quantitation can be accomplished by use of the immunogens described herein that are incorporated into an ELISA. It may also be quantitated by a competitive PCR technique. This latter technique has been used to quantitate the immunogen(s) or epitopes of the immunogen(s) of this invention.

The non-limiting examples to follow represent compositions of L. intracellularis vaccines and describe their methods of production including growing this organism in several diverse tissue culture cell lines as well as describing their use in vaccinating pigs to produce protective antibody titers and protection from challenge with virulent L. intracellularis. The examples also demonstrate the identification of target immunogens of L. intracellularis as well as the demonstration of their functionality.

The invention is further illustrated but is not intended to be limited by the following examples in which all parts and percentages are by weight unless otherwise specified.

### EXAMPLE 1

Growth of L. intracellularis in tissue culture has been routinely accomplished. One method involves growing the organism in Henle 407 cells (ATCC No. CCL6) by infecting the cells with L. intracellularis bacterial seed organisms isolated from gut homogenates of pigs exhibiting clinical proliferative ileitis. Sections of intestine from infected pigs were removed, washed to remove mucus and suspended in Hanks Balanced Salts Solution (HBSS) containing hyaluronidase to detach epithelial cells from the lamina propria. The sections were then washed and the enterocytes were harvested by centrifugation. The enterocytes were washed a second time and then were exposed to gentamycin sulfate and amphotericin B for 24 hours at 4° C. to kill contaminating gut microflora. The treated cells were harvested, washed in HBSS, and lysed with 0.5% deoxycholate for 1 hour at 37° C. With constant agitation to release the intracellular organism. The lysates were passed through a sterile 0.65 u membrane filter. Filtered lysates were added to confluent monolayers of Henle 407 human intestinal cells in 25 cm² flasks (1 ml of inoculum per flask). The infected cells were examined for cytopathic effect (CPE) during a 10 day incubation period. CPE was observed in Henle 407 cells 3 to 4 days after inoculation with enterocyte lysate. The CPE usually consisted of cell elongation, followed by rounding and sloughing of cells between days 7 and 10.

### EXAMPLE 2

L. intracellularis also has been grown on rat intestinal epithelial cells (IEC), ATCC No. 1589. Since growth of L. intracellularis on these cells does not result in CPE, the monitoring of growth was accomplished either by use of standard PCR methods or by standard fluorescent antibody detection methods using a monoclonal antibody (MoAb). Quantitation via the PCR method was measured via use of a densitometer and competitive PCR procedures known to the art.

The process of growing L. intracellularis on IEC cells comprised the steps of 1) inoculation of the IEC cells with L. intracellularis organisms; 2) incubation of the infected IEC cell culture in a media capable of supporting growth of L. intracellularis at 37° C. in the presence of atmospheric conditions which allow growth of the organisms as well as growth of the IEC cells; 3) harvesting the L. intracellularis and 4) passaging the culture at approximately ten (10) day intervals to scale up the yield of organisms.

The preferred method of inoculating L. intracellularis comprised infecting the IEC cells with L. intracellularis in suspension culture and then planting the infected suspension culture onto an attachment surface. Flasks have routinely been used as the attachment surface. However, it would be apparent to one skilled in the art that such an attachment surface can include flasks, cell cubes, cell factories, roller bottles, beads in suspension or any other type surface typically used for growth of tissue culture cells. A more preferred method of inoculating L. intracellularis comprised adding a centrifugation step after infecting the suspension culture and prior to planting the infected suspension culture onto the attachment surface.

Any media which supports growth of the IEC cells and L. intracellularis organism can be used in the incubation process. The preferred media is Minimal Essential Medium (MEM) containing serum, such as fetal bovine serum, bovine serum, calf serum, horse serum, pig serum, goat serum, sheep serum, or any other growth-enhancing sera or serum substitutes.

The preferred atmospheric conditions for growing L. intracellularis in IEC cells comprises incubating in the presence of carbon dioxide (CO₂), oxygen (O₂) and nitrogen (N₂), specifically these can be in a combination comprising 8% CO₂, 8% O₂, and 84% N₂.

Harvesting the L. intracellularis from the infected IEC cells comprised: 1) removing the medium from infected flasks; 2) removing the infected cells from the attachment surface by scraping them or treating them with art-known trypsin or trypsin/versene concentrations; and 3) releasing the L. intracellularis from the cells. Processes used for releasing the L. intracellularis from the IEC cells comprised freeze-thaw and treatment with potassium chloride (KCl). If potassium chloride is used, concentrations between 0.1 and 0.2% were used. It would be appreciated by one skilled in the art that any other treatment that releases microorganisms from tissue culture (e.g., freeze-thaw, treatment with trypsin, sonication, etc.) could be used.

The process for passaging of the L. intracellularis in IEC cells comprised repeating the above-described steps multiple times.

### EXAMPLE 3

L. intracellularis IL-B (ATCC No. 55370) was grown in IEC cells using T-25 flasks, according to the procedure described previously (Example 2). Infected IEC cells were cultured for 5 to 7 days in gaspaks in an atmosphere of CO₂:O₂:N₂ (8:8:84). Supernatant was removed and the cells were treated with 0.2% KCL for 5 min. and 0.1% KCL for 25 min. The KCL was removed and the cells were harvested by scraping. The harvested cells were passed through a 22 gauge needle to break down the cell structure. The cell lysate was subjected to low speed centrifugation for 10 min. and the semi-purified organisms remaining in the supernatant were harvested by high speed centrifugation. Antigen was pooled from 25 flasks and a portion of the antigen was subjected to a french press treatment for the production of soluble antigen. The remainder was aliquoted and stored at −70° C. This soluble antigen was formulated into a vaccine according to the following procedure. Vaccine antigen was formulated with Titermax® adjuvant or Freunds Incomplete adjuvant at a concentration of 500 ug of antigen/dose. With the Titermax® adjuvant, 0.5 mL was mixed with 0.5 mL of antigen to produce a 1.0 mL dose containing 500 ug of antigen. With the Freunds Incomplete adjuvant, 2.0 mL of adjuvant was mixed with 2.0 mL of antigen such that the total dose also contained 500 ug.

In order to determine whether the antigen produced could protect pigs from a homologous challenge or from exposure to heterologous isolates or strains, ten 4-week-old pigs were vaccinated and later challenged. Ten control pigs received equal doses of a mock vaccine which contained only the tissue culture medium Minimal Essential Medium (MEM)) and adjuvant (without antigen). The vaccine used for the first vaccination contained Titermax® adjuvant while the vaccine used for the second vaccination contained Freunds Incomplete adjuvant. Serum samples were taken prior to vaccination (prebleed), at day of booster (Day 14) and at the day of challenge (Day 35) to demonstrate the production of an immune response post vaccination. Serum was tested for antibody to L. intracellularis via an ELISA wherein the wells in a 96-well plate were coated with L. intracellularis antigen (purified from pig gut epithelial cells) of a clinical isolate which was from a different source than the isolate used to produce the vaccine. Therefore, presence of an increase in antibody response post vaccination would indicate that the vaccine strain would cross-react and thus cross-protect against infection with heterologous isolates/strains.

The pigs were numbered and than placed into two different treatment groups, four different pens, to provide two different repetitions of each treatment. The pigs were challenge exposed to L. intracellularis via intubation with 75 mL of viable L. intracellularis-infected cells per pig (5 days post cell-culture infection) 21 days after the booster vaccination. Pigs were observed for clinical signs of disease for 24 days and then necropsied and examined for lesions of ileitis (gross lesions and hyperplasia). Rectal swabs were cultured for S. hyodysenteriae and Salmonella spp. prior to challenge and at necropsy. None of these swabs were positive indicating that pigs were not infected with S. hyodysenteriae or Salmonella spp.

Table 1 shows the results of the challenge. Two pigs (one vaccinated and one control) died of respiratory lesions prior to challenge. The remaining control pigs showed sporadic diarrhea. None of the vaccinated pigs exhibited any grossly observable pathology. Upon necropsy at 24 days following challenge, seven of eight vaccinated pigs were normal, whereas, five of nine control pigs had gut lesions typical of L. intracellularis. One vaccinate had both gross lesions and hyperplasia, whereas, five control pigs showed both gross lesions and hyperplasia. One control had hyperplasia but showed no gross lesions.

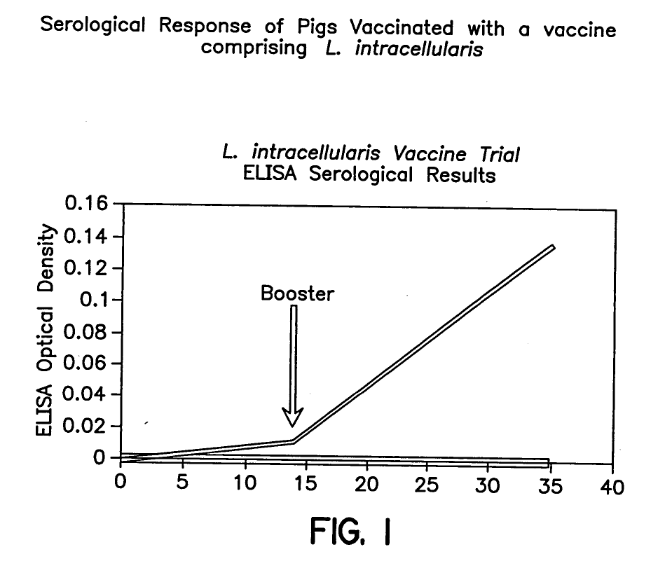

ELISA serology data (FIG. 1) indicate the development of an antibody response post primary vaccination and a significant increase in antibody response post booster. The antibody response seen in the control pigs was minimal compared to that observed in the vaccinates. This was considered to be very significant since this antibody response was measured against a heterologous field isolate of L. intracellularis. It was concluded that the vaccine stimulated a significant heterologous serological response in pigs (against a heterologous field isolate), whereas, the control pigs remained seronegative. This serological response post vaccination demonstrates the ability of the L. intracellularis prototype isolate ATCC 55370, when formulated into a vaccine, to protect pigs against field infection with heterologous isolates of this organism.

TABLE 1
Clinical and Necropsy Results of Vaccinates and Controls Post Challenge with L. intracellularis

| | | | | | Presence | | |
|---|---|---|---|---|---|---|---|
| Vaccinated | Presence of | Gross | | Control | of | Gross | |
| Pigs | Diarrhea | Lesions | Hyperplasia | Pigs | Diarrhea | Lesions | Hyperplasia |
| 6 | − | − | − | 17 | + | + | + |
| 7 | − | − | − | 10 | + | − | − |
| 11 | − | − | − | 18 | + | + | ± |
| 4 | − | − | − | 3 | + | + | + |
| 20 | − | + | ± | 8 | + | − | ± |
| 2 | − | − | − | 9 | + | − | − |
| 13 | − | − | − | 15 | + | + | ± |
| 14 | − | − | − | 1 | + | − | − |
| 12 | − | − | − | 5 | + | + | ± |

These results indicate that a vaccine comprising tissue culture grown L. intracellularis and an adjuvant protects pigs from proliferative ileitis caused by L. intracellularis.

### EXAMPLE 4

Interaction of serum from pigs recovering from proliferative ileitis with antigen extracts of L. intracellularis is one of the first methods of identifying target immunogens from an organism. The antigen used for Western blotting was prepared by growing L. intracellularis in Henle 407 cells as described previously (Example 1). Fifty micrograms of protein of either infected or uninfected cells was loaded into each well of a SDS-PAGE electrophoretic gel using a 10% acrylamide separating gel with a 4% stacking gel. The gel was run using a tris/glycine/SDS-running buffer. It was run at 30 m Amps/gel with constant voltage, constant power. The gels were run until the dye front reached the bottom of the gel, usually 4 hours.

Coumasie blue staining of the gel was done using 25% of Coomasie blue R-250 in acetic acid and methanol. The gel was put in a microwave on high for two minutes with the stain. It was then put onto a shaker at room temperature for 20 minutes. Destaining was performed using methanol and acetic acid at different concentrations. The gel was put in the microwave on high for two minutes. It was then incubated for 20 minutes on a shaker at room temperature. The destaining procedure was repeated until the background was colorless.

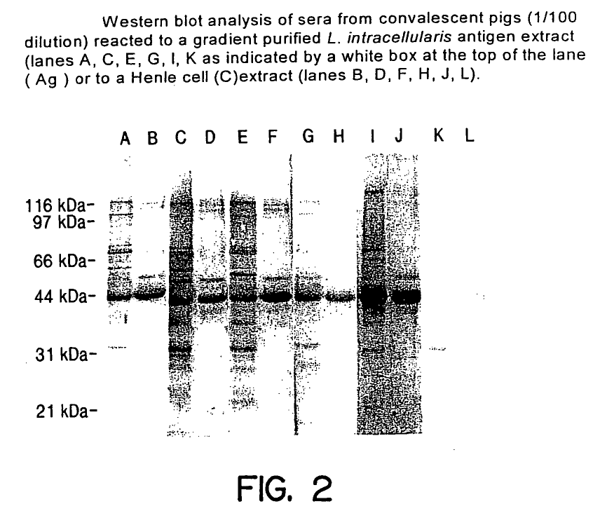

The gels were also transferred to PVDF membrane for Western blot analysis. The transfers were performed overnight at 4° C. They were run in a tris/glycine buffer at 10-15V. The membranes were then blocked with 5% nonfat milk for 30 minutes. The membranes were then incubated overnight with a 1:100 dilution of sera in 5% milk. The membranes were washed twice with PBS/tween. They were then incubated with biotin labeled goat anti-swine at a dilution of 1:200 in 5% milk for two hours. The membranes were washed again and incubated with peroxidase labeled streptavidin at a dilution of 1:400 in PBS/tween for one hour. After another wash with PBS/tween the blots were developed with 4CN/H₂O₂ for approximately 10 to 15 minutes or until good color development. The results of Western blot analysis of 6 convalescent pig sera are shown in FIG. 2. It was noted that the 43-44 kDa antigen was commonly observed in all pigs (A, C, E, G, I and K). In many of the pigs, this protein was a dominantly-recognized protein. However, in some cases, the Henle cell extract lanes (controls) also showed the presence of proteins with similar molecular weights (lanes B, D, F, H, J and L). The 21 kDa and 31 kDa antigens were routinely detected by convalescent pig sera and not by control pig sera, as were the 60 kDa and 71 kDa antigens. The ≧115 kDa antigen has also routinely been detected more consistently by the convalescent pig sera than by the control pig sera. It is concluded that these bands represent target immunogens and should be evaluated for their potential to produce neutralizing monoclonal antibodies and to produce protective vaccines.

### EXAMPLE 5

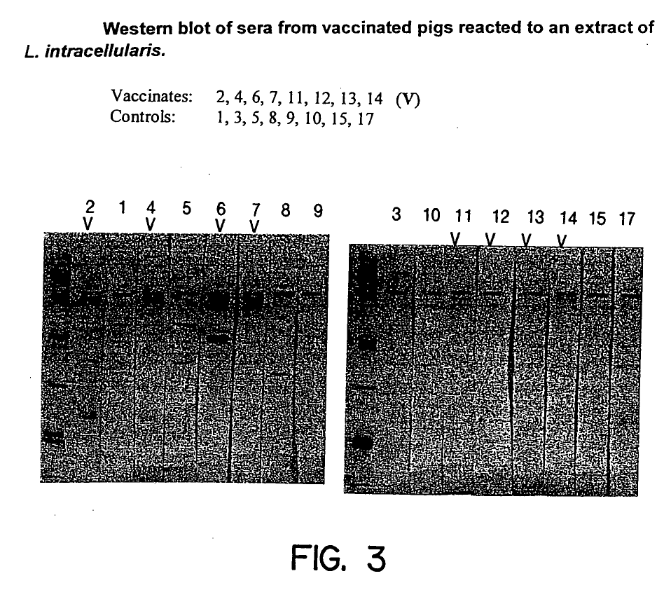

In order to further identify the target immunogens of L. intracellularis and further determine the importance of the target immunogens identified in Example 4, sera from the pigs used in Example 3 were evaluated for their ability to detect significant antigens of L. intracellularis using the Western blot analysis as described in Example 4. The L. intracellularis antigen used for the initial electrophoresis was the vaccine antigen as prepared in Example 1 prior to inactivation and adjuvanting. In this case, the pig sera was diluted 1:250 for the blotting process. The Western blots of the vaccinated and control pigs are shown in FIG. 3. It is apparent that the 43-44 kDa doublet is prominent in all of the vaccinates (Principals) and not in the Controls. The 21 kDa, 31 kDa, 41 kDa, 60 kDa, 71 kDa and ≧115 kDa bands can also be observed in the vaccinates more consistently than in the Control pigs. This confirms that pigs which have been vaccinated with a vaccine prepared according to the methods of this invention and are protected from a challenge with L. intracellularis carry antibodies to the 21 kDa, 31 kDa, 41 kDa, 43-44 kDa, 60 kDa, 71 kDa and ≧115 kDa antigens with a dominance of reactivity to the 43-44 kDa doublet. The above-listed antigens are certainly important and appear to be related to protection from L. intracellularis.

### EXAMPLE 6

A final method used to further identify the target immunogens of L. intracellularis and to determine which of the antigens of L. intracellularis were associated with antibody response in convalescent pigs (Example 4), protection in vaccinated pigs (Example 5) and which would be target immunogens for producing a subunit, recombinant or naked DNA vaccine, monoclonal antibodies were prepared against L. intracellularis and evaluated for their functionality (ability to neutralize L. intracellularis in tissue culture).

Monoclonal antibodies to L. intracellularis were produced using art-known methods. Mice were hyperimmunized with vaccine as prepared in Example 1 but grown on Henle 407 cells. The spleen from one mouse was removed using sterile techniques and placed into a petri dish with 5 mL of RPMI. The spleen's cells were dissassociated by using the plastic plunger of a sterile syringe in a petri dish. The splenocyte suspension was placed into a 15 mL conical tube and centrifuged at 1700 rpm for 5 min., after which the supernatant was removed. The pellet was suspended in 5 to 10 mL of RPMI and cells were counted. RPMI was added so that there was 1×10⁸ splenocytes per mL. One milliliter of this suspension of splenocytes was added to a tube of myeloma cells at a concentration of about 1×10⁷ and the cells were centrifuged together at 100 rpm for 5 min. The supernatant was removed and the pellet loosened. One milliliter of warm PEG mixture was added to the loosened pellet over a minute (approximately 1 drop/5 seconds). The cell suspension was allowed to remain at room temperature for 1 minute after which 21 mL of RPMI were added over a 4 minute time period using a syringe (approximately 1 drop/3 to 4 seconds). The cell suspension was centrifuged at 700 rpm for 10 minutes. The supernatant was removed and resuspended in 100 mL of thymocyte conditioned media. One milliliter of this suspension was added to each well of a 24 well plate until the entire cell suspension was dispensed. The cells growing in the 24 well plate were subjected to selection by incubating them sequentially in 2×HAT, 1×HAT and 1×HT for a period of 24 days. The hybridomas were then subcloned to isolate cells producing monoclonal antibodies which reacted with L. intracellularis. Hybridomas were selected by interacting the supernatants containing monoclonal antibodies with electrophoresed extracts from tissue culture grown L. intracellularis using standard Western blotting techniques as described in Example 4.

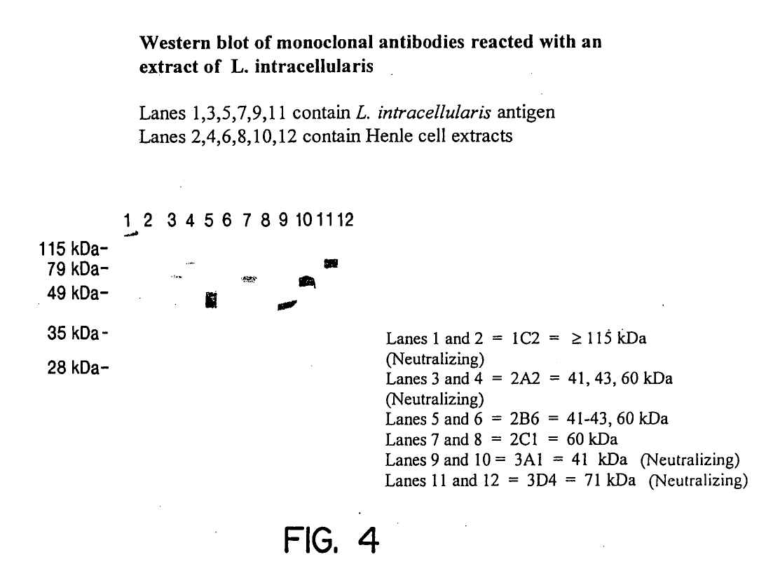

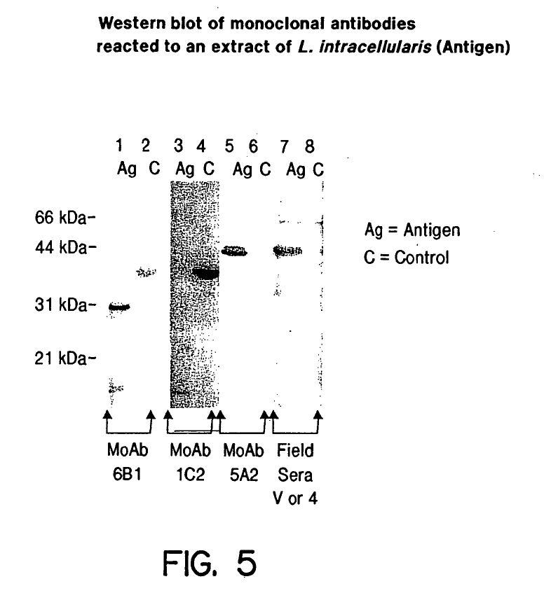

Western blot analysis was conducted using L. intracellularis antigen or non-infected cellular antigen (controls) blotted with the various monoclonal antibodies. Results of these Western blots are shown in FIGS. 4 and 5. Lanes 1, 3, 5, 7, 9 and 11 in FIG. 4 and lanes 1, 3, 5 and 7 in FIG. 5 represent Western blots of L. intracellularis antigen whereas lanes 2, 4, 6, 8, 10 and 12 in FIG. 4 and 2, 4, 6 and 8 represent the cellular antigen controls. Clone 1C2 is specific for the ≧115 kDa antigen and is shown in FIG. 4, lanes 1 and 2. Clone 2A2 detects the 60, 43 and 41 kDa bands (FIG. 4, lanes 3 and 4). Clone 2B6 also detects the 60, 43 and 41 kDa antigens, although the greatest density is observed with the 41 kDa band and the 43-44 kDa doublet (FIG. 4, lanes 5 and 6). Clone 2C1 is specific for the 60 kDa antigen (FIG. 4, lanes 7 and 8), clone 3A1 is specific for the 41 kDa antigen (FIG. 4, lanes 9 and 10), clone 3D4 is specific for the 71 kDa antigen (FIG. 4, lanes 11 and 12), and clone 5A2 is specific for the 43-44 kDa doublet (FIG. 5, lanes 5 and 6). In FIG. 5, lanes 7 and 8 represent reaction of the antigen extract with convalescent sera designated Vor 4.

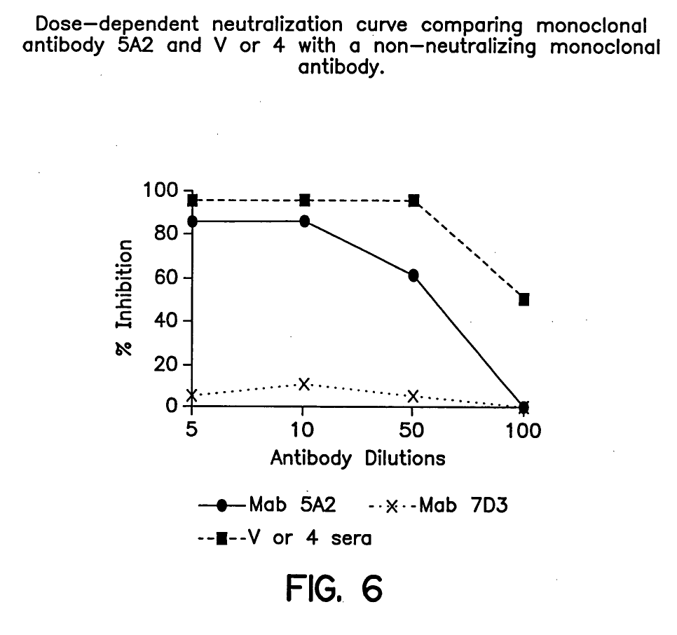

These monoclonal antibodies were then tested for their ability to neutralize CPE observed in Henle cell cultures infected with L. intracellularis. Results of the neutralization test for clone 5A2 are compared with neutralization by a field sera (Vor 4) and a non-neutralizing MoAb (Mab 7D3) in FIG. 6. Mab 5A2 demonstrates a significant neutralization of the L. intracellularis CPE. MoAbs from clones 2A2, 3D4, 1C2 and 3A1 demonstrated similar neutralization of L. intracellularis CPE.

Therefore, the target immunogens of L. intracellularis with molecular weights of ≧115, 71 60 and 43-44, 41, 31 and 21 kDa have been identified. One or more of these target immunogens are expected to produce a satisfactory subunit, recombinant or naked DNA vaccines as well as successful antigens for use in diagnosing proliferative ileitis and quantitate antigens during vaccine production. Monoclonal antibodies when prepared specifically against these antigens have demonstrated the ability to neutralize L. intracellularis in culture or appear to be very immunogenic and essential in the development of immunity to the L. intracellularis.

Although the invention has been described in detail in the foregoing for the purpose of illustration, it is to be understood that such detail is solely for that purpose and that variations can be made therein by those skilled in the art without departing from the spirit and scope of the invention except as it may be limited by the claim.

1. A proliferative ileitis vaccine comprising an immunogenically effective amount of isolated Lawsonia intracellularis 41 kDa, 43 kDa and 44 kDa antigens.

2. (canceled)

3. The proliferative ileitis vaccine according to claim 1 further comprising an adjuvant.

4. (canceled)

5. The proliferative ileitis vaccine according to claim 3, comprising at lease one adjuvant is selected from the group consisting of an polymer, oil in water emulsion, a water-in-oil-in-water emulsion, a lipid, aluminum hydroxide, aluminum phosphate, aluminum sulfate, an immunomodulator and combinations thereof.

6-21. (canceled)

22. A method for protecting swine from disease caused by L. intracellulars, comprising administering to said swine an effective amount of the vaccine according to claim 1.

23-26. (canceled)
